(19) United States
(12) Patent Application Publication
(10) Pub. No.: US 2023/0248450 A1
(43) Pub. Date: Aug. 10, 2023

(54) DISPLAY SYSTEMS FOR ROBOTIC SURGICAL SYSTEMS

(71) Applicant: EndoQuest Robotics, Inc., Houston, TX (US)

(72) Inventors: Jayavardhan Ravi, Stafford, TX (US); Seoungkyou Lee, Houston, TX (US); Jiwon Choi, Houston, TX (US); Dongsuk Shin, Houston, TX (US)

(73) Assignee: EndoQuest Robotics, Inc., Houston, TX (US)

(21) Appl. No.: 18/134,392

(22) Filed: Apr. 13, 2023

Related U.S. Application Data
(63) Continuation of application No. PCT/US2022/051246, filed on Nov. 29, 2022.
(60) Provisional application No. 63/284,125, filed on Nov. 30, 2021.

(51) Int. Cl. A61B 34/00; G16H 40/63; G06F 3/0481; A61B 34/37

(52) U.S. Cl. CPC A61B 34/25; G16H 40/63; G06F 3/0481; A61B 34/37; A61B 34/74; A61B 2090/373

(57) ABSTRACT

A display system for a robotic surgical system can include a display module configured to display a graphical user interface (GUI) on a display for providing information relating to one or more medical instruments. The GUI can include an image display area for displaying an image from a videoscope extending from an overtube, a first instrument simulator configured to provide a first simulated representation of one or more medical instruments extending from the overtube from a first elevation view, and a second instrument simulator configured to provide a second simulated representation of the one or more medical devices extending from the overtube from a second elevation view different than the first elevation view.

### CROSS-REFERENCE TO RELATED APPLICATIONS

This application is a continuation of International Patent Application No. PCT/US2022/051246 filed Nov. 29, 2022, which claims priority to and the benefit of U.S. Provisional Application No. 63/284,125 filed Nov. 30, 2021, the entire contents of which are herein incorporated by reference in their entirety.

### FIELD

This disclosure relates to robotic surgical systems, e.g., for minimally invasive surgery including, but not limited to, endoluminal and single-site surgery.

### BACKGROUND

Minimally invasive surgery such as endoluminal and single-site robotic surgery offer significant advantages versus traditional robotic surgery. For example, in endoluminal robotic surgery, no incision need be made to access difficult to access locations within a patient's natural lumen. This dramatically reduces and/or eliminates recovery time and improves procedural safety. A single-site system reduces incisions to a minimum single-site, which reduces an otherwise larger number of incisions to provide access for certain procedures.

Certain endoluminal and single-site robotic surgical systems have been proposed. Examples of such systems and related components can be found in U.S. Pat. No. 10,881,422, as well as U.S. Patent Application Nos. US20210322046, US20210322045, US20190117247, US20210275266, US20210267702, US20200107898, US20200397457, US202000397456, US20200315645, and US201962914226, all of the above being incorporated by reference herein in their entirety.

Conventional surgical robotics and systems have generally been considered satisfactory for their intended purpose. However, there is still a need in the art for improved robotic surgical systems, devices, methods, controls, and components, especially those configured for endoluminal and single-site surgery. The present disclosure provides improvements in such areas, for example.

### SUMMARY

In accordance with at least one aspect of this disclosure, a display system for a robotic surgical system can include a display module configured to display a graphical user interface (GUI) on a display for providing information relating to one or more medical instruments. The GUI can include an image display area for displaying an image from a videoscope extending from an overtube, a first instrument simulator configured to provide a first simulated representation of one or more medical instruments extending from the overtube from a first elevation view, and a second instrument simulator configured to provide a second simulated representation of the one or more medical devices extending from the overtube from a second elevation view different than the first elevation view.

The GUI can include an instrument control indicator including at least one of an instrument name indicator, a connection state indicator for indicating a connection of the instrument to a hub, an attachment state indicator for indicating an attachment of the instrument to a robotic instrument controller, an instrument lifespan indicator, and/or one or more instrument specific operation indicators. The GUI can include a system clutch indicator for indicating whether a system clutch is active for disengaging operation of one or more medical instruments, the videoscope and/or the overtube of the system. The GUI can include a finger clutch indicator for indicating whether a finger clutch is pressed for disengaging operation of the one or more medical instruments associated with the finger clutch. The GUI can include a notification indicator for displaying notifications to the user, wherein the notification indicator is centered and at the bottom of the display.

The GUI can include an overtube translation indicator configured to indicate an amount of translation of an overtube and/or whether the translation is within one or more overtube translation limits. The GUI can include an overtube roll indicator for the overtube and configured to indicate an amount of roll of the overtube and/or whether the roll of the overtube is within one or more overtube roll limits. The GUI can include an overtube flex indicator for the overtube and configured to indicate an amount of flex of the overtube in a horizontal and/or vertical plane, and/or whether the flex of the overtube is within one or more overtube flex limits. The GUI can include a videoscope flex indicator for the videoscope and configured to indicate an amount of flex of the videoscope in a horizontal and/or vertical plane, and/or whether the flex of the videoscope is within one or more videoscope flex limits. The GUI can include a videoscope shape indicator for the videoscope, which can be configured to indicate an amount of flex of a distal segment and an amount of flex of a proximal segment of the videoscope.

The GUI can include an instrument roll indicator for each medical instrument configured to indicate an amount of instrument roll and/or whether the instrument roll is within one or more limits. The GUI can include an instrument translation indicator for each medical instrument configured to indicate an amount of instrument translation and/or whether the instrument translation is within one or more instrument translation limits.

In accordance with at least one aspect of this disclosure, a robotic surgical system can include a user console comprising a display and one or more input devices. The robotic surgical system can include any suitable embodiment of a display system disclosed herein, e.g., described above.

In accordance with at least one aspect of this disclosure, a method can include calculating a position and/or orientation of a robotically controlled elongated surgical device, and generating one or more representative images of the elongated surgical device showing a position and/or orientation of the elongated surgical device relative to a neutral position. Calculating a position and/or orientation can include using instrument controller information to deduce a position and/or orientation of the elongated surgical device. In certain embodiments, generating one or more representative images can include generating a plurality of 2-dimensional indicators configured to indicate one or more of pitch, yaw, and roll.

In certain embodiments, the method can include generating a color indicator that changes color based on a relative position of the one or more representative images to one or more limits away from neutral. The color indicator can be associated with the representative image of the elongated device. The color indicator can be positioned adjacent to or around the representative image.

These and other features of the embodiments of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, embodiments thereof will be described in detail herein below with reference to certain figures, wherein:

FIG. 1 is a perspective view of an embodiment of a system in accordance with this disclosure;

FIGS. 2A and 2B illustrate an embodiment of a graphical user interface in accordance with this disclosure;

FIG. 3A illustrates an embodiment of a first instrument simulator in accordance with this disclosure;

FIG. 3B illustrates an embodiment of a second instrument simulator in accordance with this disclosure;

FIG. 4 illustrates an embodiment of an instrument control indicator in accordance with this disclosure;

FIGS. 5A, 5B, 5C, and 5D illustrate an embodiment of one or more instrument control indicators in various states in accordance with this disclosure;

FIG. 6 illustrates an embodiment of a finger clutch indicator in accordance with this disclosure;

FIG. 7 illustrates an embodiment of a system clutch indicator in accordance with this disclosure;

FIG. 8 illustrates an embodiment of a notification indicator in accordance with this disclosure, showing various states;

FIG. 9A illustrates an embodiment of an overtube translation indicator in accordance with this disclosure;

FIG. 9B illustrates the embodiment of FIG. 9A in context with an embodiment of user input and system output in accordance with this disclosure;

FIG. 10A illustrates an embodiment of an overtube roll indicator in accordance with this disclosure;

FIG. 10B illustrates the embodiment of FIG. 10A in context with an embodiment of a clockwise user input and system output in accordance with this disclosure;

FIG. 10C illustrates the embodiment of FIG. 10A in context with an embodiment of a counterclockwise user input and system output in accordance with this disclosure;

FIG. 11A illustrates an embodiment of an overtube flex indicator in accordance with this disclosure, showing various states;

FIGS. 11B and 11C illustrate the embodiment of FIG. 11A in context with an embodiment of user input and system output in accordance with this disclosure;

FIG. 12A illustrates an embodiment of a videoscope flex indicator in accordance with this disclosure, showing various states;

FIGS. 12B and 12C illustrate the embodiment of FIG. 12A in context with an embodiment of user input and system output in accordance with this disclosure;

FIG. 13 illustrates an embodiment of an instrument roll indicator in accordance with this disclosure, showing various states; and

FIG. 14 illustrates an embodiment of a videoscope shape indicator in accordance with this disclosure, showing various states.

### DETAILED DESCRIPTION

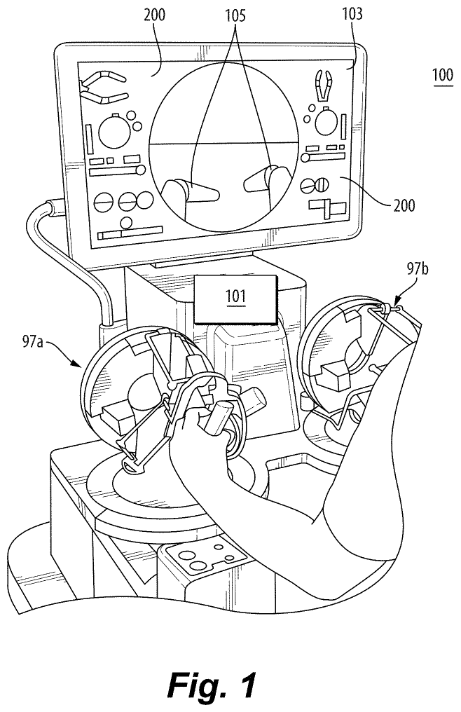

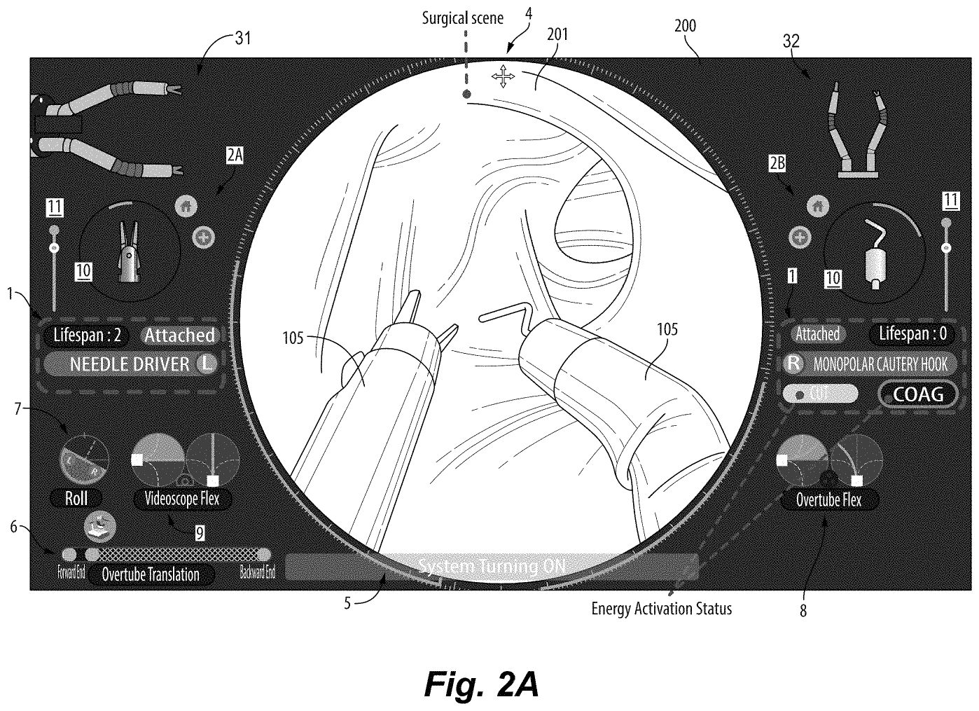

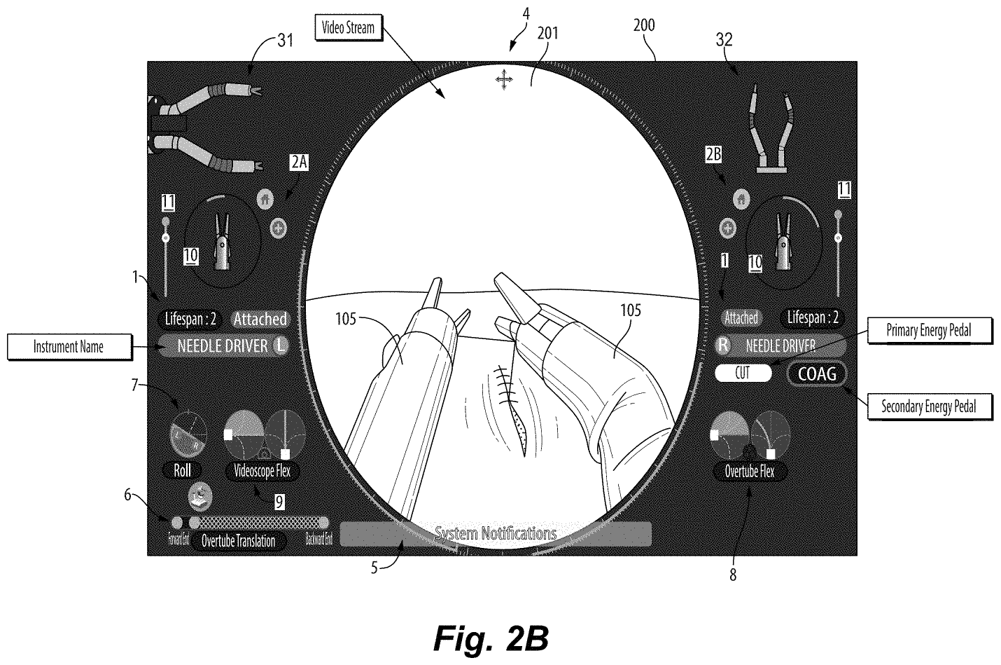

Reference will now be made to the drawings wherein like reference numerals identify similar structural features or aspects of the subject disclosure. For purposes of explanation and illustration, and not limitation, an illustrative view of an embodiment of a graphical user interface (GUI) in accordance with the disclosure is shown in FIG. 1 and is designated generally by reference character 100. Other embodiments and/or aspects of this disclosure are shown in FIGS. 2A-14.

In accordance with at least one aspect of this disclosure, referring to FIGS. 1-14, a display system 100 for a robotic surgical system (e.g., an endoluminal robotic system) can include a display module 101 configured to display a graphical user interface (GUI) 200 on a display 103 for providing information relating to one or more medical instruments 105 (e.g., robotically controlled endoscopic tools). FIG. 1 is a perspective view of an embodiment of a system in accordance with this disclosure. The display module 101 can be operatively connected to any suitable feedback systems, control modules, inputs, and/or outputs of a robotic surgical system to receive any data associated with the GUI 200 and/or the indicators disclosed herein (e.g., described below). Any module(s) disclosed herein can be or include any suitable hardware and/or software module(s) configured to perform any suitable function (e.g., as disclosed herein).

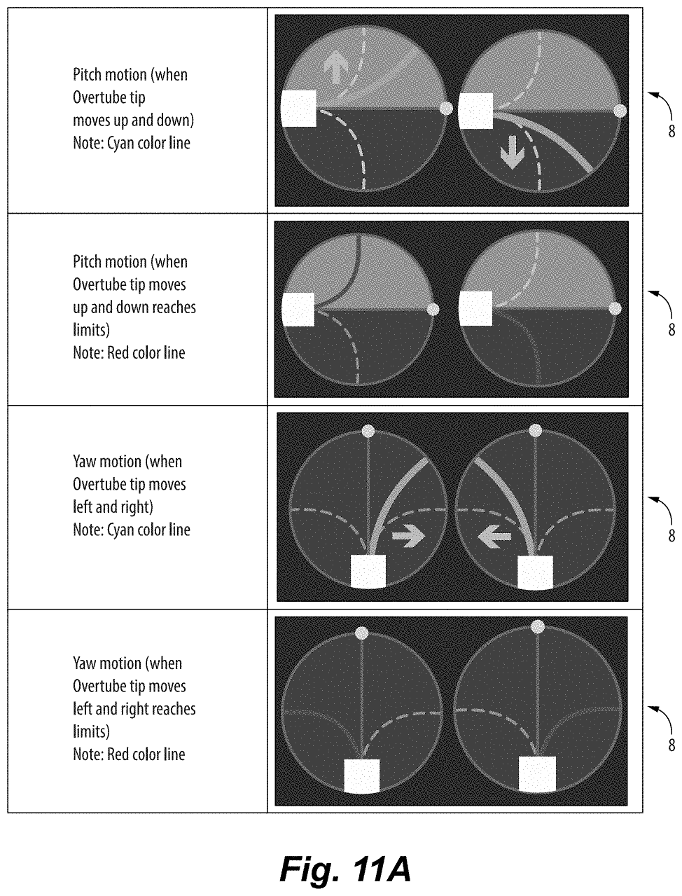

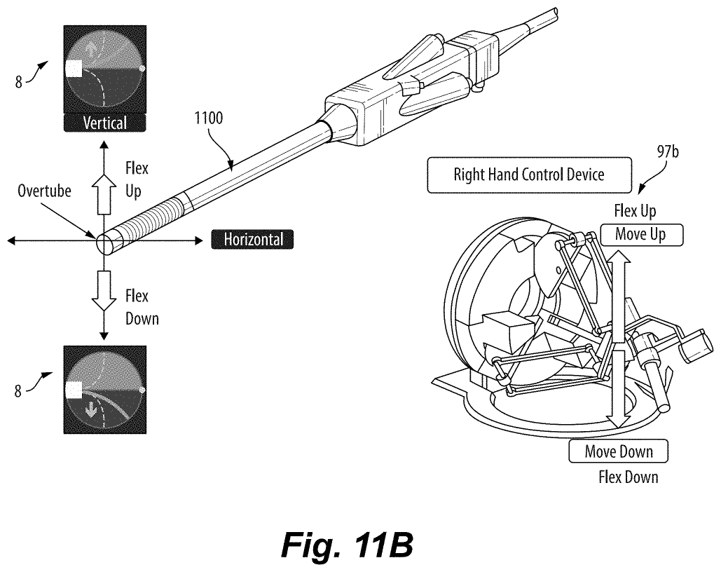

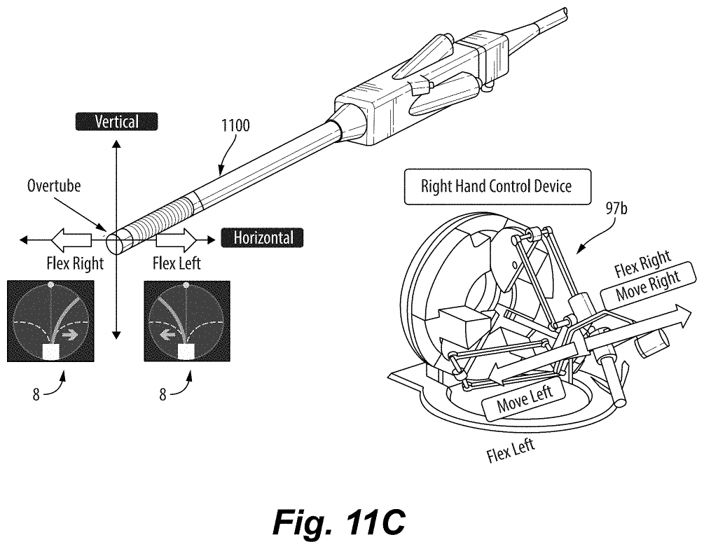

FIGS. 2A and 2B illustrate an embodiment of a graphical user interface in accordance with this disclosure. The GUI 200 can include an image display area 201 (e.g., depicted as the surgical scene in FIG. 2A or the video stream in FIG. 2B) for displaying an image from a videoscope 1200 extending from an overtube 1100 which can include a flexible elongated insertion tube having one or more instrument channels therein for the medical device to advance/retract therein (e.g., as shown in FIGS. 11B and 11C).

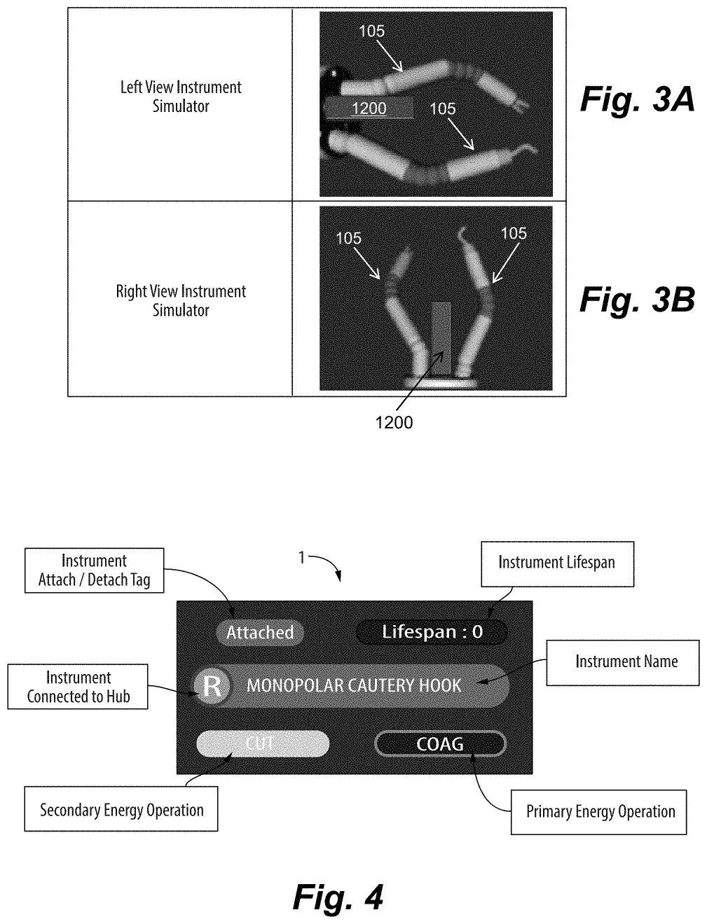

Referring to FIGS. 2A-3B, the GUI 200 can also include a first instrument simulator 31 configured to provide a first simulated representation of one or more medical instruments 105 extending from the overtube 1100 from a first elevation view (e.g., left view instrument simulator as shown in FIG. 3A), and a second instrument simulator 32 configured to provide a second simulated representation of the one or more medical devices 105 extending from the overtube 1100 from a second elevation view different than the first elevation view (e.g., orthogonal views such as a right view instrument simulator of FIG. 3B). Any suitable relative views are contemplated herein.

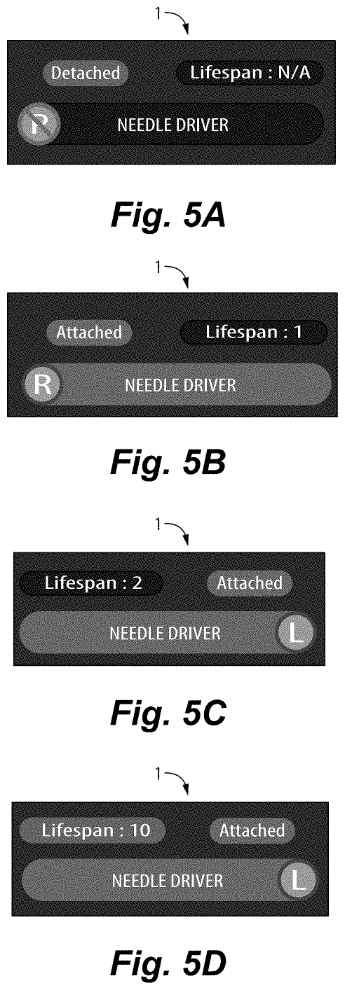

Referring additionally to FIGS. 4-5D, the GUI 200 can include an instrument control indicator 1 including, e.g., at least one of an instrument name indicator, a connection state indicator for indicating a connection of the instrument to a hub, an attachment state indicator for indicating an attachment of the instrument to a robotic instrument controller, an instrument lifespan indicator, and/or one or more instrument specific operation indicators (e.g., primary and secondary energy operation as shown). Any suitable instrument control indicators are contemplated herein. In certain embodiments, the instrument lifespan can be fixed (e.g., a fixed number of uses of the device determined objectively). In certain embodiments, the instrument lifespan can be variable depending on one or more physical factors (e.g., actual/objectively determined wear) and/or manufacturers' requirements, for example.

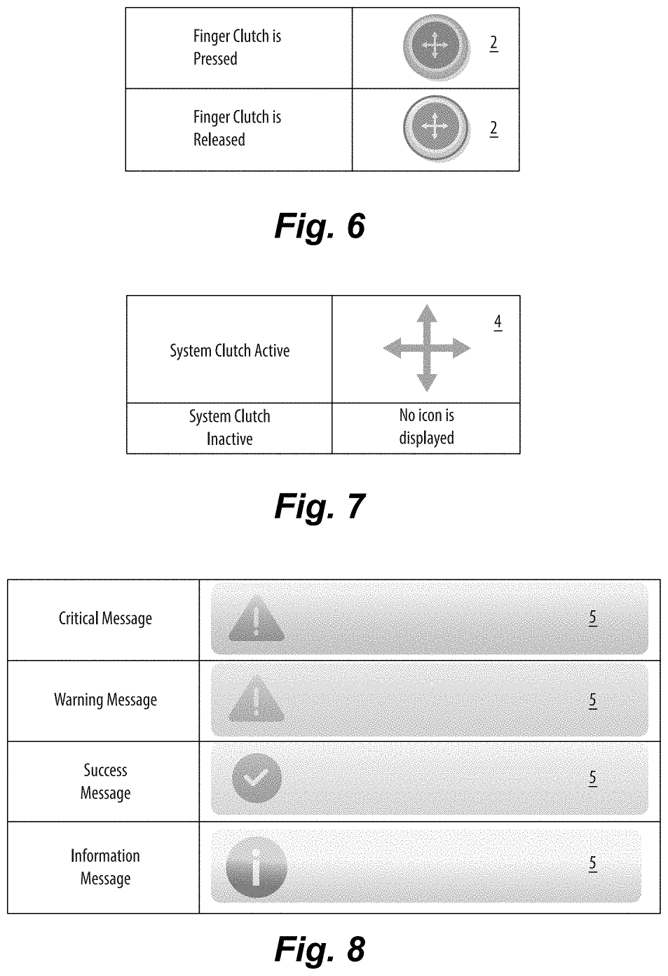

Referring additionally to FIG. 6, the GUI 200 can include a finger clutch indicator 2 for indicating whether a finger clutch is pressed for disengaging operation of the one or more medical instruments 105 associated with the finger clutch. Referring additionally to FIG. 7, the GUI 200 can include a system clutch indicator 4 for indicating whether a system clutch is active for disengaging operation of the one or more medical instruments 105, the overtube 1100 and/or the videoscope 1200 of the system.

Referring to FIG. 8, the GUI 200 can include a notification indicator 5 for displaying notifications to the user, wherein the notification indicator is centered and at the bottom of the display. The notification indicator 5 can include a less than full opacity, and/or can change colors as a function of message content, e.g., as shown.

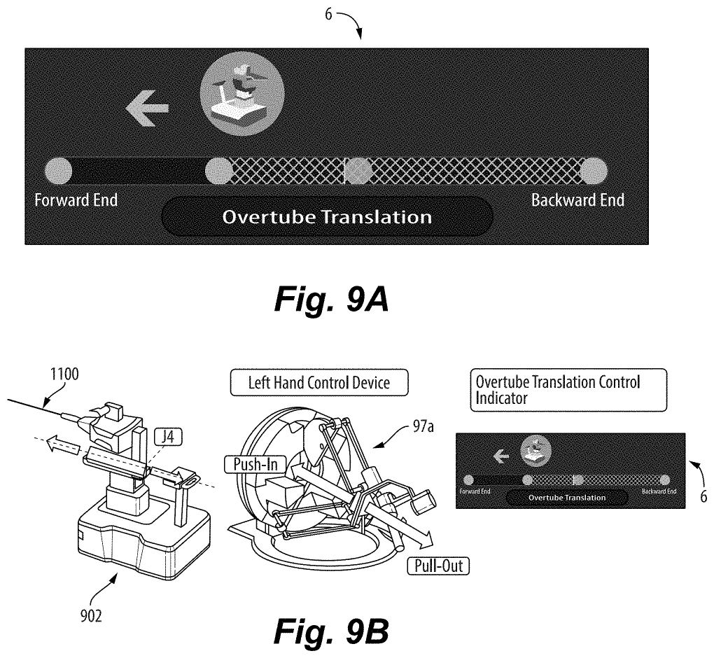

Referring additionally to FIGS. 9A and 9B, the GUI 200 can include an overtube translation indicator 6 configured to indicate an amount of translation (e.g., axial limits) of an overtube 1100 (shown attached to a mobile patient cart/console 902) and/or whether the translation is within one or more overtube translation limits (e.g., shown limits as depicted). Any suitable color coded change in the indicators as a function of proximity to the limits is contemplated herein.

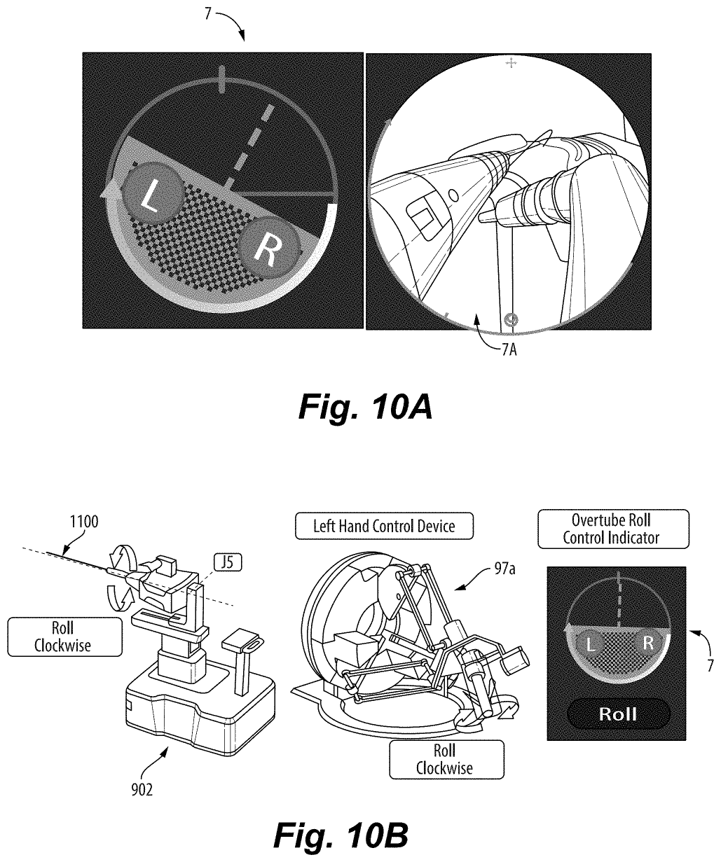

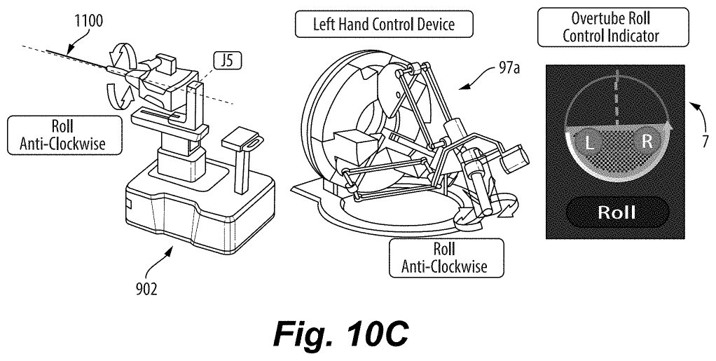

Referring additionally to FIGS. 10A-10C, the GUI 200 can include an overtube roll indicator 7 for the overtube 1100 and configured to indicate an amount of roll of the overtube 1100 and/or whether the roll of the overtube 1100 is within one or more overtube roll limits (e.g., color coded as a function of percentage of minimum and maximum roll). In certain embodiments, the image display area 201 can include a supplemental roll indicator 7A at an edge of the image, e.g., as shown.

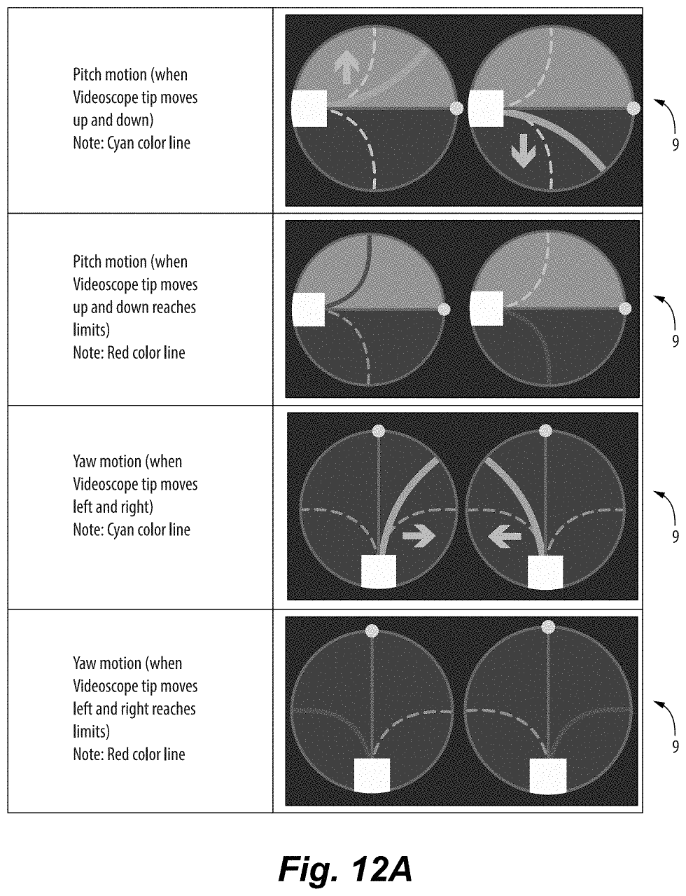

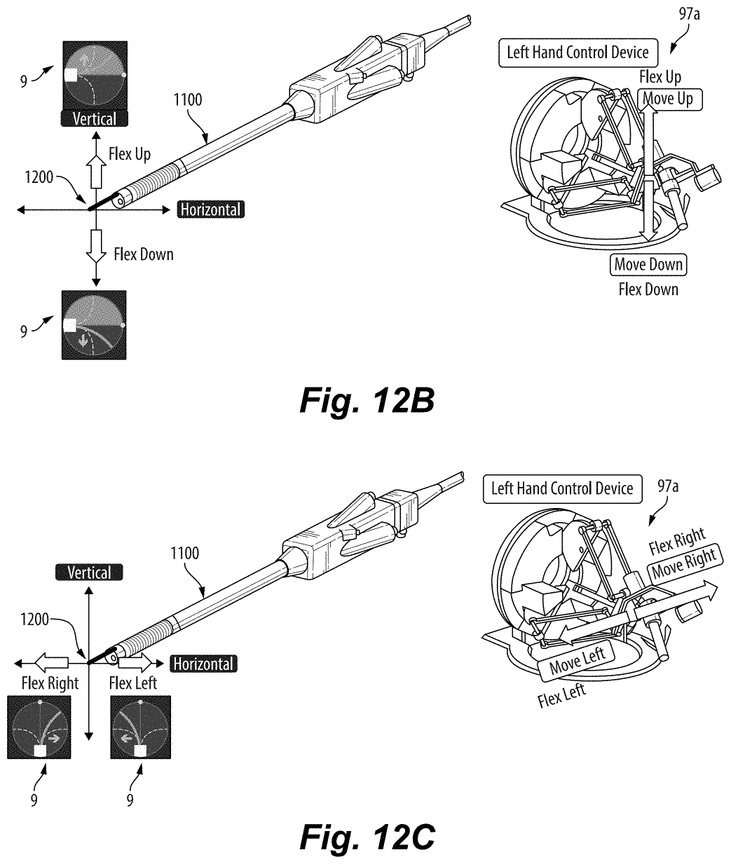

Referring additionally to FIGS. 11A-11C, the GUI 200 can include an overtube flex indicator 8 for the overtube 1100 and configured to indicate an amount of flex of the overtube 1100 in a horizontal and/or vertical plane (e.g., both as shown), and/or whether the flex of the overtube 1100 is within one or more overtube flex limits (e.g., color coded as a function of percentage of minimum and maximum flex). Referring additionally to FIGS. 12A-12C, the GUI 200 can include a videoscope flex indicator 9 for the videoscope 1200 and configured to indicate an amount of flex of the videoscope 1200 in a horizontal and/or vertical plane (e.g., both as shown), and/or whether the flex of the videoscope 1200 is within one or more videoscope flex limits (e.g., color coded as a function of percentage of minimum and maximum flex).

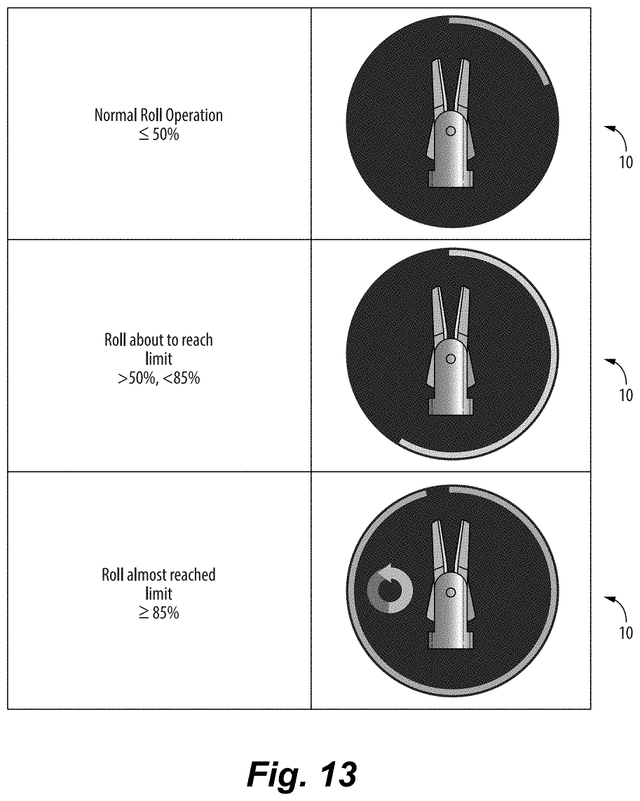

Referring additionally to FIG. 13, the GUI 200 can include an instrument roll indicator for each medical instrument 105 configured to indicate an amount of instrument roll and/or whether the instrument roll is within one or more limits (e.g., color coded as a function of percentage of minimum and maximum roll). The instrument roll indicator 10 can be a line that surrounds an image (simulated or real) of the attached medical instrument 105. For example, the line can progressively fill the more roll that is present, and can change color as a function of percentage of fill (e.g., blue, to yellow, to orange, to red). The GUI 200 can include an instrument translation indicator 11 for each medical instrument configured to indicate an amount of instrument translation and/or whether the instrument translation is within one or more instrument translation limits (e.g., color coded as a function of percentage of minimum and maximum translation).

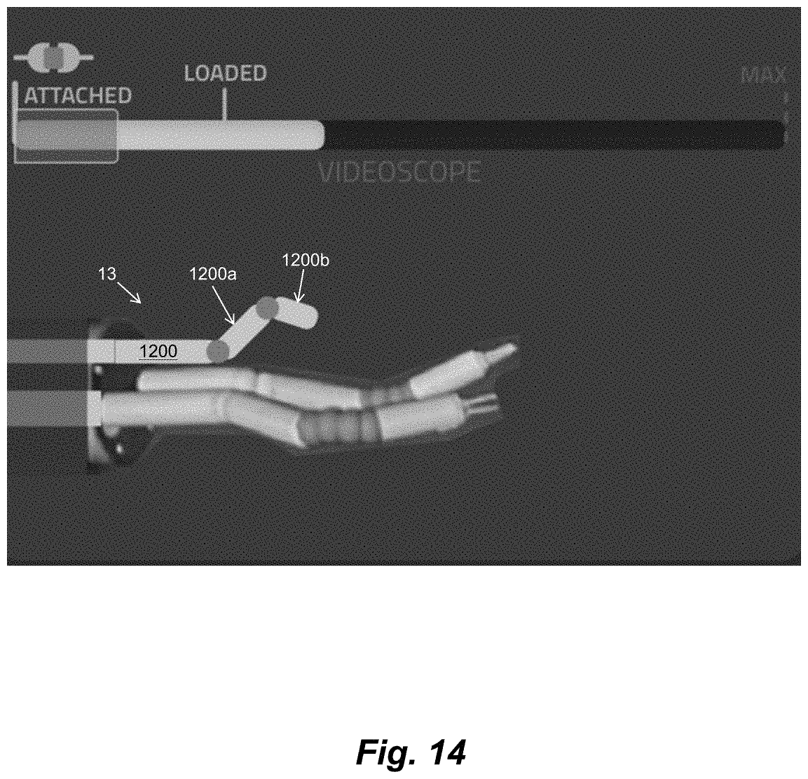

Referring additionally to FIG. 14, the GUI 200 can include a videoscope shape indicator 13 for the videoscope 1200, which can also be configured to indicate an amount of flex of a distal segment 1200b and an amount of flex of a proximal segment 1200a of the videoscope 1200. In certain embodiments, the videoscope indicator 13 can be integrated with the first instrument simulator 31 and/or second instrument simulator 32, for example (e.g., as shown). In certain embodiments, the videoscope indicator 13 can be separate from the first instrument simulator 31 and/or second instrument simulator 32, for example. In certain embodiments, the videoscope shape indicator 13 can be not only configured to provide a simulated representation of the videoscope in a flex state (e.g., in a cobra shape as shown), but also configured to provide a simulated line indicating the amount of flex of a distal segment and an amount of flex of a proximal segment of the videoscope 1200.

In accordance with at least one aspect of this disclosure, a robotic surgical system (e.g., an endoluminal robotic surgery system) can include a user console 99 comprising a display and one or more input devices 97a, 97b (e.g., left hand control device and/or right hand control device). The robotic surgical system can include any suitable embodiment of a display system disclosed herein, e.g., system 100 as described above.

In accordance with at least one aspect of this disclosure, a method can include calculating a position and/or orientation of a robotically controlled elongated surgical device, and generating one or more representative images of the elongated surgical device showing a position and/or orientation of the elongated surgical device relative to a neutral position. Calculating a position and/or orientation can include using instrument controller information to deduce a position and/or orientation of the elongated surgical device. In certain embodiments, generating one or more representative images can include generating a plurality of 2-dimensional indicators configured to indicate one or more of pitch, yaw, and roll.

In certain embodiments, the method can include generating a color indicator that changes color based on a relative position of the one or more representative images to one or more limits away from neutral. The color indicator can be associated with the representative image of the elongated device. The color indicator can be positioned adjacent to or around the representative image.

Embodiments of a GUI 200 can include the relative position of indicators shown having an advantageous ergonomic position. Embodiments of a GUI 200 can include the shapes of each indicator shown which can have functional advantages. Any suitable combination of indicators, and/or other arrangements and/or shapes are contemplated herein.

Certain embodiments of a GUI and/or indicators are disclosed above. Any other suitable GUI and/or indicators therefor is contemplated herein.

As will be appreciated by those skilled in the art, aspects of the present disclosure may be embodied as a system, method or computer program product. Accordingly, aspects of this disclosure may take the form of an entirely hardware embodiment, an entirely software embodiment (including firmware, resident software, micro-code, etc.), or an embodiment combining software and hardware aspects, all possibilities of which can be referred to herein as a “circuit,” “module,” or “system.” A “circuit,” “module,” or “system” can include one or more portions of one or more separate physical hardware and/or software components that can together perform the disclosed function of the “circuit,” “module,” or “system”, or a “circuit,” “module,” or “system” can be a single self-contained unit (e.g., of hardware and/or software). Furthermore, aspects of this disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of this disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of this disclosure may be described above with reference to flowchart illustrations and/or block diagrams of methods, apparatus (systems) and computer program products according to embodiments of this disclosure. It will be understood that each block of any flowchart illustrations and/or block diagrams, and combinations of blocks in any flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in any flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified herein.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the subject disclosure includes reference to certain embodiments, those skilled in the art will readily appreciate that changes and/or modifications may be made thereto without departing from the spirit and scope of the subject disclosure.

What is claimed is:

1. A display system for a robotic surgical system, comprising:
a display module configured to display a graphical user interface (GUI) on a display for providing information relating to one or more medical instruments, the GUI comprising:
an image display area for displaying an image from a videoscope extending from an overtube;
a first instrument simulator configured to provide a first simulated representation of one or more medical instruments extending from the overtube from a first elevation view; and
a second instrument simulator configured to provide a second simulated representation of the one or more medical devices extending from the overtube from a second elevation view different than the first elevation view.

2. The system of claim 1, wherein the GUI includes an instrument control indicator including at least one of an instrument name indicator, an instrument lifespan indicator, and/or one or more instrument specific operation indicators.

3. The system of claim 1, wherein the GUI includes a system clutch indicator for indicating whether a system clutch is active for disengaging operation of the one or more medical instruments, the videoscope and/or the overtube of the system.

4. The system of claim 1, wherein the GUI includes a finger clutch indicator for indicating whether a finger clutch is pressed for disengaging operation of the one or more medical instruments associated with the finger clutch.

5. The system of claim 1, wherein the GUI includes a notification indicator for displaying notifications to the user, wherein the notification indicator is centered and at the bottom of the display.

6. The system of claim 1, wherein the GUI includes an overtube translation indicator configured to indicate an amount of translation of an overtube and/or whether the translation is within one or more overtube translation limits.

7. The system of claim 1, wherein the GUI includes an overtube roll indicator for the overtube and configured to indicate an amount of roll of the overtube and/or whether the roll of the overtube is within one or more overtube roll limits.

8. The system of claim 1, wherein the GUI includes an overtube flex indicator for the overtube and configured to indicate an amount of flex of the overtube in a horizontal and/or vertical plane, and/or whether the flex of the overtube is within one or more overtube flex limits.

9. The system of claim 1, wherein the GUI includes a videoscope flex indicator for the videoscope and configured to indicate an amount of flex of the videoscope in a horizontal and/or vertical plane, and/or whether the flex of the videoscope is within one or more videoscope flex limits.

10. The system of claim 1, wherein the GUI includes a videoscope shape indicator for the videoscope and configured to indicate an amount of flex of a distal segment and an amount of flex of a proximal segment of the videoscope.

11. The system of claim 1, wherein the GUI includes an instrument roll indicator for each medical instrument configured to indicate an amount of instrument roll and/or whether the instrument roll is within one or more instrument roll limits.

12. The system of claim 1, wherein the GUI includes an instrument translation indicator for each medical instrument configured to indicate an amount of instrument translation and/or whether the instrument translation is within one or more instrument translation limits.

13. A robotic surgical system, comprising:
a user console comprising a display and one or more input devices; and
a display system for a robotic surgical system, comprising:
a display module configured to display a graphical user interface (GUI) on the display for providing information relating to one or more medical instruments, the GUI comprising:
an image display area for displaying an image from a videoscope extending from an overtube;
a first instrument simulator configured to provide a first simulated representation of one or more medical instruments extending from the overtube from a first elevation view; and
a second instrument simulator configured to provide a second simulated representation of the one or more medical devices extending from the overtube from a second elevation view different than the first elevation view.

14. The system of claim 12, wherein the GUI includes an instrument control indicator including at least one of an instrument name indicator, a connection state indicator for indicating a connection of the instrument to a hub, an attachment state indicator for indicating an attachment of the instrument to a robotic instrument controller, an instrument lifespan indicator, and/or one or more instrument specific operation indicators.

15. The system of claim 12, wherein the GUI includes a system clutch indicator for indicating whether a system clutch is active for disengaging operation of all medical instruments of the system.

16. The system of claim 12, wherein the GUI includes a finger clutch indicator for indicating whether a finger clutch is pressed for disengaging operation of the one or more medical instruments associated with the finger clutch.

17. The system of claim 12, wherein the GUI includes a notification indicator for displaying notifications to the user, wherein the notification indicator is centered and at the bottom of the display.

18. The system of claim 12, wherein the GUI includes an overtube translation indicator configured to indicate an amount of translation of an overtube and/or whether the translation is within one or more overtube translation limits.

19. The system of claim 12, wherein the GUI includes an overtube roll indicator for the overtube and configured to indicate an amount of roll of the overtube and/or whether the roll of the overtube is within one or more overtube roll limits.

20. A method for controlling a robotic surgical system, comprising:
calculating a position and/or orientation of a robotically controlled elongated surgical device; and
generating one or more representative images of the elongated surgical device showing a position and/or orientation of the elongated surgical device relative to a neutral position.

21. The method of claim 19, wherein calculating a position and/or orientation comprises using instrument controller information to deduce a position and/or orientation of the elongated surgical device.

22. The method of claim 20, wherein generating one or more representative images comprises generating a plurality of 2-dimensional indicators configured to indicate one or more of pitch, yaw, and roll.

23. The method of claim 21, further comprising generating a color indicator that changes color based on a relative position of the one or more representative images to one or more limits away from neutral.

24. The method of claim 22, wherein the color indicator is associated with the representative image of the elongated device.

25. The method of claim 23, wherein the color indicator is position adjacent to or around the representative image.
